(19) United States
(12) Patent Application Publication
(10) Pub. No.: US 2026/0307046 A1
(43) Pub. Date: Oct. 8, 2026

(54) CARBON DIOXIDE ABSORBENT, METHOD FOR RECOVERING CARBON DIOXIDE, AND APPARATUS FOR SEPARATING AND RECOVERING CARBON DIOXIDE

(71) Applicant: MITSUBISHI GAS CHEMICAL COMPANY, INC., Tokyo (JP)

(72) Inventors: Kazuki KOUNO, Kanagawa (JP); Yuki KAWASHIMA, Kanagawa (JP); Takuya SUGITA, Niigata (JP); Aoto YAMAICHI, Niigata (JP)

(73) Assignee: MITSUBISHI GAS CHEMICAL COMPANY, INC., Tokyo (JP)

(21) Appl. No.: 19/481,302

(22) PCT Filed: Jun. 27, 2024
(86) PCT No.: PCT/JP2024/023421

§ 371 Date: Nov. 4, 2025

(30) Foreign Application Priority Data
Jul. 6, 2023 (JP) 2023-111437

(51) Int. Cl. B01J 20/22; B01D 53/14

(52) U.S. Cl. CPC B01J 20/22; B01D 53/1475; B01D 2252/204; B01D 2257/504; B01J 2220/46

(57) ABSTRACT

Provided are a carbon dioxide absorbent comprising a non-azo solid amine compound (A) having two or more aromatic rings and having two or more primary amino groups, a method for capturing carbon dioxide using the carbon dioxide absorbent, and a carbon dioxide separation and capture apparatus.

### TECHNICAL FIELD

The present invention relates to a carbon dioxide absorbent, a method for capturing carbon dioxide, and a carbon dioxide separation and capture apparatus.

### BACKGROUND ART

From a viewpoint of global warming issues, there is a need to reduce carbon dioxide. One of methods for reducing carbon dioxide is a technology of efficiently capturing a high concentration (about 10 to 30% by volume) of carbon dioxide from exhaust gas discharged from thermal power plants and the like, and burying and storing it in the ground or in the sea (CCS: carbon dioxide capture and storage). Technologies related to carbon dioxide absorbents for use in CCS include, for example, those described in Patent Literatures 1 and 2.

Patent Literature 1 discloses a method for capturing carbon dioxide using a specific alkanolamine as a carbon dioxide absorbent. Patent Literature 2 discloses that a carbon dioxide absorption liquid containing carbon dioxide chemoabsorbent amine having a nitrogen-hydrogen bond and a tertiary amine solvent having no nitrogen-hydrogen bond is used as a carbon dioxide absorbent.

As described in Patent Literatures 1 and 2, many technologies using alkanolamines are known about carbon dioxide absorbents for use in CCS.

By contrast, a technology of directly capturing a low concentration of carbon dioxide (about 0.04% by volume) from the air (DAC: direct air capture) has received attention in recent years. Carbon dioxide absorbents for use in DAC are required to have higher carbon dioxide absorption capacity than that of carbon dioxide absorbents for use in CCS.

Technologies related to DAC have been variously studied. For example, Patent Literature 3 discloses an absorbent for absorbing carbon dioxide from the air, containing alkylamine substituted by a hydroxy group or an optionally substituted amino group.

Patent Literature 4 discloses a carbon dioxide absorbent containing a predetermined amount or more of a polyamine compound having an alicyclic hydrocarbon structure.

Patent Literature 5 discloses a carbon dioxide absorbent containing a predetermined amount or more of an amine compound having a predetermined heterocyclic structure.

Patent Literature 6 discloses a carbon dioxide absorbent containing a specific cyclic amine compound and a porous material.

### CITATION LIST

### Patent Literature

PTL1: JP 2008-13400 A
PTL2: JP 2017-104776 A
PTL3: JP 2017-031046 A
PTL4: WO 2022/138302 A
PTL5: WO 2022/163209 A
PTL6: WO 2023/013397 A

### SUMMARY OF INVENTION

### Technical Problem

The carbon dioxide absorbent disclosed in Patent Literature 6 is a solid absorbent having an amine compound supported on a porous material.

Usually, in reusing a solid absorbent, a steam stripping method or the like is used for desorbing carbon dioxide from the absorbent after absorption of carbon dioxide. However, in the case of using the amine compounds disclosed in Patent Literatures 1 to 6, the amine compounds might be eluted from the porous material during steam stripping, or the amine compounds might volatilize due to heating during steam stripping. Thus, conventionally known carbon dioxide absorbents are still susceptible to further improvement in repeated usability.

An object of the present invention is to provide a carbon dioxide absorbent excellent in carbon dioxide absorption property and repeated usability, a method for capturing carbon dioxide using the same, and a carbon dioxide separation and capture apparatus.

### Solution to Problem

The present inventors have found that a carbon dioxide absorbent containing a specific solid amine compound can attain the above object.

That is, the present invention relates to the following.

[1]

A carbon dioxide absorbent comprising a non-azo solid amine compound (A) having two or more aromatic rings and having two or more primary amino groups.

[2]

The carbon dioxide absorbent according to [1], wherein the amine compound (A) is a non-polymer compound.

[3]

The carbon dioxide absorbent according to [1] or [2], wherein the amine compound (A) is an amine compound represented by the following general formula (1):

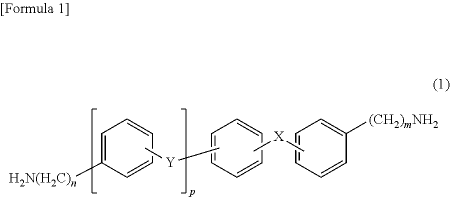

wherein in the formula (1), X and Y each independently represent a single bond or an oxygen atom; n and m each independently represent a number of 1 to 6; and p represents a number of 0 to 2.
[4]

The carbon dioxide absorbent according to [3], wherein the amine compound represented by the general formula (1) is a compound represented by any of the following formulas (1-1) to (1-3):

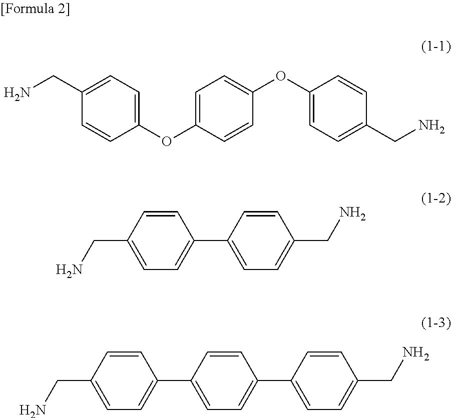

[5]

The carbon dioxide absorbent according to any one of [1] to [4], wherein the carbon dioxide absorbent comprises the amine compound (A) and a porous material (B), and at least some of the amine compound (A) is supported on the porous material (B).

[6]

The carbon dioxide absorbent according to [5], wherein the porous material (B) comprises at least one selected from the group consisting of porous silica and porous alumina.

[7]

A method for capturing carbon dioxide, comprising using the carbon dioxide absorbent according to any one of [1] to [6].

[8]

The method according to [7], wherein the method comprises:
an absorption step of bringing a gas containing carbon dioxide into contact with the carbon dioxide absorbent, to cause the carbon dioxide absorbent to absorb the carbon dioxide, and
a desorption step of desorbing carbon dioxide from the carbon dioxide absorbent with carbon dioxide absorbed in the absorption step, and
wherein the desorption step comprises at least one step selected from the group consisting of the following (I) to (III):
(I) a step of subjecting the carbon dioxide absorbent with carbon dioxide absorbed to a reduced pressure condition;
(II) a step of bringing an inert gas not containing carbon dioxide into contact with the carbon dioxide absorbent with carbon dioxide absorbed; and
(III) a step of heating the carbon dioxide absorbent with carbon dioxide absorbed.
   [9]

A carbon dioxide separation and capture apparatus comprising:
an absorption apparatus comprising a mechanism for bringing a gas containing carbon dioxide into contact with the carbon dioxide absorbent according to any one of [1] to [6], to cause the carbon dioxide absorbent to absorb the carbon dioxide; and
a desorption apparatus comprising a mechanism for desorbing carbon dioxide from the carbon dioxide absorbent with carbon dioxide absorbed.

### Advantageous Effects of Invention

According to the present invention, a carbon dioxide absorbent excellent in carbon dioxide absorption property and repeated usability, a method for capturing carbon dioxide using the same, and a carbon dioxide separation and capture apparatus can be provided.

### BRIEF DESCRIPTION OF DRAWING

FIG. 1 is a schematic diagram showing an embodiment of a carbon dioxide separation and capture apparatus of the present invention.

FIG. 2 is a graph showing results of evaluating a carbon dioxide absorption property using carbon dioxide absorbents of Examples 1 to 6.

### DESCRIPTION OF EMBODIMENTS

### Definition

Embodiments of the present invention (hereinafter, also simply referred to as “the present embodiments”) will be described in detail. The following embodiments are examples to explain the present invention and do not limit the contents of the present invention. The present invention can be implemented with appropriate modifications within the scope of the invention. In the present embodiments, it is considered that provisions that are preferred can be adopted arbitrarily, and combinations of preferred ones are more preferred. In the present embodiments, description of “XX to YY” means “XX or more and YY or less”.

In the present specification, “good carbon dioxide absorption property” means a large in the amount of carbon dioxide absorbed by a carbon dioxide absorbent. “Good absorption property for carbon dioxide from the air” means a large in the amount absorbed for carbon dioxide at a low concentration (about 0.04% by volume) from the air.

In the present specification, “excellent repeated usability” means both a high mass retention rate of a carbon dioxide absorbent and a high retention rate of an amount of carbon dioxide absorbed when cycle tests of absorption and desorption of carbon dioxide are performed.

### [Carbon Dioxide Absorbent]

A carbon dioxide absorbent of the present invention comprises a non-azo solid amine compound (A) having two or more aromatic rings and having two or more primary amino groups (hereinafter, also simply referred to as “amine compound (A)”).

The carbon dioxide absorbent of the present invention, by having the above configuration, is excellent in carbon dioxide absorption property and repeated usability. The carbon dioxide absorbent of the present invention is particularly excellent in repeated usability even in the case of using a steam stripping method in desorbing carbon dioxide from the absorbent after absorption of carbon dioxide.

The carbon dioxide absorbent of the present invention exhibits a good absorption property for carbon dioxide from the air having a carbon dioxide concentration as low as about 0.04% by volume. Accordingly, the carbon dioxide absorbent of the present invention can be suitably used in technology of directly absorbing carbon dioxide (DAC) from the air.

The carbon dioxide absorbent of the present invention can be also suitably used in case of capturing carbon dioxide from a gas having a carbon dioxide concentration as low as, e.g., 0.01% by volume or more and 1% by volume or less.

The reason why the carbon dioxide absorbent of the present invention exerts the above effects is not certain, but is considered to be as follows.

The amine compound (A) contained in the carbon dioxide absorbent has a structure having aromatic rings and having two or more primary amino groups, and the amino groups easily form carbonate through reaction with carbon dioxide and are expected to exert an excellent carbon dioxide absorption property.

The amine compound (A), by having two or more aromatic rings, easily becomes solid amine through elevation in melting point of the amine compound (A), while its solubility in water is decreased. This improves the repeated usability of the carbon dioxide absorbent. Particularly, the amine compound (A) is difficult to elute even in the case of using a steam stripping method in desorbing carbon dioxide after absorption of carbon dioxide, and the resulting carbon dioxide absorbent is expected to be excellent in repeated usability. Furthermore, the amine compound (A) is required to be a non-azo compound from the viewpoint of being applied to a method using a heating approach such as a steam stripping method in desorbing carbon dioxide, and avoiding degeneration caused by photoreaction.

### <Amine Compound (A)>

The amine compound (A) is a non-azo solid amine compound having two or more aromatic rings and having two or more primary amino groups.

In the present specification, “primary amino group” means an amino group having two hydrogen atoms on a nitrogen atom, i.e., —NH₂ group.

In the present specification, “non-azo compound” means a compound not containing an azo bond (—N═N—) in the molecule.

In the present specification, “solid amine compound” means an amine compound that has a melting point of higher than 25° C. and is a solid at 25° C.

The melting point of the amine compound (A) is preferably 50° C. or more, more preferably 80° C. or more, even more preferably 100° C. or more, still more preferably 110° C. or more, and yet more preferably 120° C. or more, from the viewpoint of improving the repeated usability. An upper limit is not limited, but is preferably 300° C. or less, more preferably 280° C. or less, even more preferably 250° C. or less, still more preferably 200° C. or less, yet more preferably 180° C. or less, even much more preferably 160° C. or less, and still much more preferably 150° C. or less, from the viewpoint of improving the carbon dioxide absorption property and from the viewpoint of improving the supporting property of a porous material (B) described later.

Specifically, the melting point of the amine compound (A) can be measured by a method described in Examples.

The amine compound (A) has two or more aromatic rings from the viewpoint of improving the carbon dioxide absorption property and the repeated usability. The number of aromatic rings in the amine compound (A) is preferably 2 or more and 10 or less, more preferably 2 or more and 6 or less, even more preferably 2 or more and 4 or less, still more preferably 2 or more and 3 or less, and yet more preferably 2, from the viewpoint of improving the carbon dioxide absorption property and the repeated usability and from the viewpoint of the ease of desorption of carbon dioxide.

The number of amino groups in the amine compound (A) is 2 or more from the viewpoint of improving the carbon dioxide absorption property, and is preferably 8 or less, more preferably 6 or less, even more preferably 4 or less, and still more preferably 3 or less, from the viewpoint of improving the repeated usability by reducing water solubility.

The number of primary amino groups in the amine compound (A) is 2 or more from the viewpoint of improving the carbon dioxide absorption property, and is preferably 8 or less, more preferably 6 or less, even more preferably 4 or less, and still more preferably 3 or less, from the viewpoint of improving the repeated usability by reducing water solubility.

The amine compound (A) has preferably 30% by mol or more, more preferably 40% by mol or more, even more preferably 50% by mol or more, still more preferably 60% by mol or more, yet more preferably 70% by mol or more, even much more preferably 80% by mol or more, and still much more preferably 90% by mol or more of a primary amino group and has 100% by mol or less of a primary amino group, with respect to all amino groups contained in the amine compound (A) from the viewpoint of improving the carbon dioxide absorption property.

The amine compound (A) is preferably a non-polymer compound from the viewpoint of improving the carbon dioxide absorption property and the repeated usability and from the viewpoint of improving the supporting property of a porous material (B) described later.

In the present specification, “non-polymer compound” means a low-molecular compound and specifically means a compound having a molecular weight of 1,000 or less. The molecular weight of the amine compound (A) is preferably 800 or less, more preferably 700 or less, even more preferably 600 or less, still more preferably 500 or less, yet more preferably 400 or less, and even much more preferably 350 or less from the viewpoint of improving the carbon dioxide absorption property and from the viewpoint of improving the supporting property of a porous material (B), and is preferably 212 or more from the viewpoint of improving the repeated usability.

A maximum carbon dioxide release temperature of the amine compound (A) is preferably 280° C. or less, more preferably 250° C. or less, even more preferably 200° C. or less, still more preferably 180° C. or less, and yet more preferably 150° C. or less, from viewpoints of improving the carbon dioxide desorption performance and further improving the repeated usability. A lower limit value of the above maximum carbon dioxide release temperature is not limited, but is, for example, 40° C. or more, preferably 80° C. or more, even more preferably 100° C. or more.

The maximum carbon dioxide release temperature is a value measured by the following method.

The amine compound (A) is heated with carbon dioxide absorbed from 23° C. to 250° C. at 10° C./minute of a heating rate, and a temperature at which an endothermic amount associated with desorption of carbon dioxide reaches a maximum is measured, and this temperature is taken as the maximum carbon dioxide release temperature. Here, the amine compound (A) with carbon dioxide absorbed can be prepared, for example, by allowing 5 mmol of the amine compound (A) to stand in the air at 23° C. and 50% RH for 24 hours.

The amine compound (A) is preferably an amine compound represented by the following general formula (1) from the viewpoint of improving the carbon dioxide absorption property and the repeated usability.

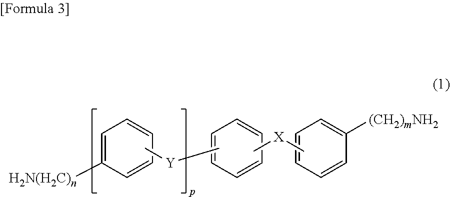

In the formula (1), X and Y each independently represent a single bond or an oxygen atom. n and m each independently represent a number of 1 to 6, and p represents a number of 0 to 2.

X and Y in the formula (1) may be the same or may be different, but are preferably the same from the viewpoint of the ease of production. That is, both of X and Y in the formula (1) are preferably single bonds or oxygen atoms.
n and m in the formula (1) may be the same or may be different, but are preferably the same from the viewpoint of the ease of production. Each of n and m is preferably 1 to 4, more preferably 1 to 3, even more preferably 1 to 2, and still more preferably 1, from the viewpoint of improving the carbon dioxide absorption property.
p in the formula (1) is preferably 0 or 1, and more preferably 0, from the viewpoint of improving the carbon dioxide absorption property and the repeated usability.

When p is 2, each of X and Y in the formula (1) is preferably an oxygen atom.

The amine compound represented by the general formula (1) is preferably a compound represented by any of the following formulas (1-1) to (1-3) from the viewpoint of improving the carbon dioxide absorption property and the repeated usability and from the viewpoint of the ease of production, more preferably a compound represented by the following formula (1-1) or (1-2) from the viewpoint of improving the carbon dioxide absorption property, and even more preferably a compound represented by the following formula (1-2).

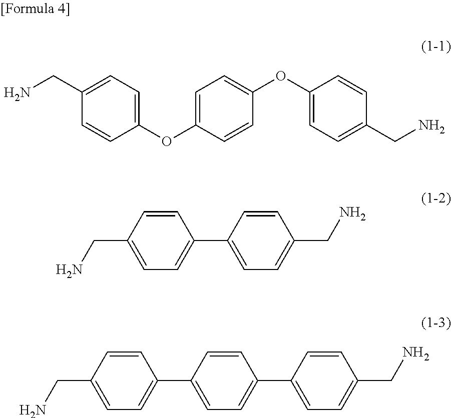 [SMILES: NCc1ccc(Oc2ccc(Oc3ccc(CN)cc3)cc2)cc1 ; NCc1ccc(-c2ccc(CN)cc2)cc1 ; NCc1ccc(-c2ccc(-c3ccc(CN)cc3)cc2)cc1]

One of these amine compounds (A) can be used alone, or two or more thereof can be used in combination.

The amine compound (A) can be produced by a known method, and a commercially available product may be used.

A content of the amine compound (A) in all amine compounds in the carbon dioxide absorbent of the present invention is preferably 50% by mass or more, more preferably 60% by mass or more, even more preferably 70% by mass or more, still more preferably 80% by mass or more, yet more preferably 90% by mass or more, even much more preferably 95% by mass or more, and still much more preferably 98% by mass or more, and 100% by mass or less, from the viewpoint of improving the carbon dioxide absorption property and the repeated usability.

The content of the amine compound (A) in the carbon dioxide absorbent of the present invention is preferably 10% by mass or more, more preferably 20% by mass or more, even more preferably 30% by mass or more, still more preferably 40% by mass or more, yet more preferably 45% by mass or more, and even much more preferably 50% by mass or more, and 100% by mass or less, from the viewpoint of improving the carbon dioxide absorption property. When the carbon dioxide absorbent comprises a porous material (B) described later, the content of the amine compound (A) in the carbon dioxide absorbent is preferably 90% by mass or less, more preferably 80% by mass or less, and even more preferably 70% by mass or less, from the viewpoint of improving the carbon dioxide absorption property.

Preferably, the carbon dioxide absorbent of the present invention comprises the amine compound (A) and a porous material (B), and at least some of the amine compound (A) is supported on the porous material (B), from the viewpoint of further improving the carbon dioxide absorption property and from the viewpoint that carbon dioxide can be desorbed with lower energy after carbon dioxide absorption.

The carbon dioxide absorbent comprises the amine compound (A) and a porous material (B), and at least some of the amine compound (A) is supported on the porous material (B), whereby the carbon dioxide absorption property is improved. This is presumably because the surface area of a portion, of the solid amine compound (A), capable of coming into contact with carbon dioxide is widened. Depending on the type of the amine compound (A), the binding state between the amine compound (A) and carbon dioxide is changed so that the maximum carbon dioxide release temperature of the carbon dioxide absorbent is lower than that of the amine compound (A) alone. Therefore, it is expected that carbon dioxide can be desorbed under a lower energy condition.

### <Porous Material (B)>

The porous material (B) is preferably one that can support the amine compound (A) and withstand conditions of capturing carbon dioxide, and includes, for example, at least one selected from the group consisting of silica, alumina, silica-alumina, magnesia, zirconia, zeolite, a zeolite analogous compound, a clay mineral, a natural mineral, activated carbon, a carbon molecular sieve (porous carbon), porous resin (synthetic adsorbent), a metal organic structure, and a waste solid. Among these, the porous material (B) preferably comprises at least one selected from the group consisting of the silica and the alumina, more preferably comprises the silica, and even more preferably comprises mesoporous silica, from the viewpoint of further improving the carbon dioxide absorption property and from the viewpoint that carbon dioxide can be desorbed with lower energy after carbon dioxide absorption.

A content of the silica or the alumina, preferably the silica, and more preferably the mesoporous silica, in the porous material (B) is preferably 50% by mass or more, more preferably 60% by mass or more, even more preferably 70% by mass or more, still more preferably 80% by mass or more, yet more preferably 90% by mass or more, even much more preferably 95% by mass or more, and still much more preferably 98% by mass or more, and 100% by mass or less, from the viewpoint of further improving the carbon dioxide absorption property and from the viewpoint that carbon dioxide can be desorbed with lower energy after carbon dioxide absorption.

A shape of the porous material (B) is preferably in a particulate form. This can increase a specific surface area of the porous material (B) and improve an amount of the amine compound (A) supported, thereby further improving the amount of carbon dioxide absorbed in the carbon dioxide absorbent.

A volume median particle size (D₅₀) of the porous material (B) measured by laser diffraction/scattering particle size distribution measurement is preferably 1 μm or more, more preferably 5 μm or more, and even more preferably 10 μm or more, from a viewpoint of improving handling, and preferably 500 μm or less, more preferably 300 μm or less, even more preferably 200 μm or less, still more preferably 180 μm or less, and yet more preferably 160 μm or less, from a viewpoint of further improving the amount of the amine compound (A) supported.

The specific surface area of the porous material (B) measured by a BET method is preferably 2 m²/g or more, more preferably 10 m²/g or more, even more preferably 100 m²/g or more, still more preferably 200 m²/g or more, yet more preferably 400 m²/g or more, and even much more preferably 600 m²/g or more, from the viewpoint of further improving the amount of the amine compound (A) supported, and preferably 3,000 m²/g or less, more preferably 1,500 m²/g or less, even more preferably 1,200 m²/g or less, and still more preferably 1,000 m²/g or less, from the viewpoint of improving the handling.

The pore volume of the porous material (B) is preferably 0.1 cm³/g or more, more preferably 0.3 cm³/g or more, and even more preferably 0.5 cm³/g or more, from the viewpoint of further improving the amount of the amine compound (A) supported, and preferably 5.0 cm³/g or less, more preferably 3.0 cm³/g or less, even more preferably 2.5 cm³/g or less, still more preferably 2.0 cm³/g or less, yet more preferably 1.5 cm³/g or less, and even much more preferably 1.0 cm³/g or less, from the viewpoint of improving the repeated usability.

The specific surface area and the pore volume can be measured, for example, by a specific surface area/pore size distribution measurement analyzer (e.g., “ASAP2020” produced by Shimadzu Corporation) using a constant volume method. As a more specific gas adsorption measurement method using the specific surface area/pore size distribution measurement analyzer, for example, a sample is pretreated by heating and vacuum evacuation, and 0.1 g of the sample for measurement is placed in a sample tube. The sample is then heated to 40° C., vacuum evacuated for 6 hours, cooled to room temperature, and mass of the sample is measured. In the measurement, a liquid nitrogen temperature is set and a pressure range is specified, and the specific surface area, the pore volume, and the pore size can be analyzed and calculated from the obtained nitrogen adsorption isotherms.

The porous material (B) may be granulation molded from the particulate porous material by a known method to make pellets, tablets, or other granulated products. The granulation molding method includes, for example, a dry granulation method using a compression molding machine and a wet granulation method using a binder. By using the particulate porous material granulation molded, vibration resistance and abrasion resistance can be imparted, thereby improving physical stability.

When the carbon dioxide absorbent comprises the amine compound (A) and the porous material (B), a content of the amine compound (A) in the carbon dioxide absorbent is preferably 10 parts by mass or more, more preferably 20 parts by mass or more, even more preferably 30 parts by mass or more, still more preferably 40 parts by mass or more, yet more preferably 50 parts by mass or more, even much more preferably 70 parts by mass or more, and still much more preferably 80 parts by mass or more, and preferably 1000 parts by mass or less, more preferably 500 parts by mass or less, even more preferably 250 parts by mass or less, still more preferably 200 parts by mass or less, yet more preferably 150 parts by mass or less, and even much more preferably 120 parts by mass or less, with respect to 100 parts by mass of the porous material (B) from the viewpoint of further improving the carbon dioxide absorption property and from the viewpoint that carbon dioxide can be desorbed with lower energy after carbon dioxide absorption.

When the carbon dioxide absorbent comprises the amine compound (A) and the porous material (B), a total content of the amine compound (A) and the porous material (B) in the carbon dioxide absorbent is preferably 60% by mass or more, more preferably 70% by mass or more, even more preferably 80% by mass or more, still more preferably 90% by mass or more, yet more preferably 95% by mass or more, and even much more preferably 98% by mass or more, and 100% by mass or less, when the total amount of the carbon dioxide absorbent is 100% by mass from the viewpoint of further improving the carbon dioxide absorption property and from the viewpoint that carbon dioxide can be desorbed with lower energy after carbon dioxide absorption.

### <Other Components>

The carbon dioxide absorbent may include components other than the amine compound (A), and the porous material (B) as appropriate to the extent that the effects of the invention are not impaired. The components other than the amine compound (A) and the porous material (B) include, for example, a deterioration inhibitor, an antifoaming agent, an antioxidant, and a desiccant to remove moisture (magnesium sulfate, molecular sieves, etc.).

A content of water in the carbon dioxide absorbent is, from the viewpoint of improving the carbon dioxide absorption property and the repeated usability, preferably 10% by mass or less, more preferably 5% by mass or less, even more preferably 1% by mass or less, still more preferably 0.5% by mass or less, yet more preferably 0.1% by mass or less, and even much more preferably 0.01% by mass or less, and it is still much more preferable that the carbon dioxide absorbent is substantially free of water. Here, “substantially free of water” means that water is not intentionally added and does not exclude the presence of a small amount of water as an impurity.

When the carbon dioxide absorbent comprises the amine compound (A) and the porous material (B), a melting point of the carbon dioxide absorbent is preferably 50° C. or more, more preferably 80° C. or more, even more preferably 100° C. or more, still more preferably 110° C. or more, and yet more preferably 120° C. or more, from the viewpoint of improving the repeated usability. An upper limit is not limited, but is preferably 300° C. or less, more preferably 280° C. or less, even more preferably 250° C. or less, still more preferably 200° C. or less, yet more preferably 180° C. or less, even much more preferably 160° C. or less, and still much more preferably 150° C. or less, from the viewpoint of improving the carbon dioxide absorption property.

The melting point of the carbon dioxide absorbent can be measured by the same method as above.

When the carbon dioxide absorbent comprises the amine compound (A) and the porous material (B), a maximum carbon dioxide release temperature of the carbon dioxide absorbent is preferably 200° C. or less, more preferably 180° C. or less, even more preferably 160° C. or less, and still more preferably 150° C. or less, from viewpoints of improving the carbon dioxide desorption performance and further improving the repeated usability. A lower limit value of the above maximum carbon dioxide release temperature is not limited, but is, for example, 40° C. or more, preferably 80° C. or more, even more preferably 100° C. or more.

The maximum carbon dioxide release temperature can be measured by the same method as above.

From the viewpoint that carbon dioxide can be desorbed at a temperature lower than the melting point of the carbon dioxide absorbent, the melting point of the carbon dioxide absorbent is preferably a temperature of higher than the maximum carbon dioxide release temperature of the carbon dioxide absorbent.

It is also preferable that the melting point of the carbon dioxide absorbent and the maximum carbon dioxide release temperature of the carbon dioxide absorbent differ largely. If the difference is small between the melting point of the carbon dioxide absorbent and the maximum carbon dioxide release temperature of the carbon dioxide absorbent, the carbon dioxide absorbent might be melted and aggregated at the same time with the desorption of carbon dioxide to decrease the surface area, resulting in a reduced carbon dioxide absorption property. However, when the difference is large from the maximum carbon dioxide release temperature of the carbon dioxide absorbent, carbon dioxide can be desorbed at a temperature lower than the melting point of the carbon dioxide absorbent. In addition, the reduction in carbon dioxide absorption property caused by the melting of the carbon dioxide absorbent can be circumvented.

The difference between the melting point and the maximum carbon dioxide release temperature of the carbon dioxide absorbent (the melting point of the carbon dioxide absorbent−the maximum carbon dioxide release temperature of the carbon dioxide absorbent) is preferably 5° C. or more, more preferably 10° C. or more, and even more preferably 20° C. or more, from the viewpoint of further improving the carbon dioxide absorption property and from the viewpoint that carbon dioxide can be desorbed with lower energy after carbon dioxide absorption.

When the carbon dioxide absorbent comprises the amine compound (A) and the porous material (B), a method for preparing the carbon dioxide absorbent is not limited, and any known method can be used. For example, the amine compound (A) and the porous material (B) can be blended, and mixed using a known apparatus to prepare it.

When the carbon dioxide absorbent is in an aspect in which at least some of the amine compound (A) is supported on the porous material (B), the carbon dioxide absorbent can preferably be prepared by the following method.

First, the amine compound (A) is dissolved in an organic solvent, and the porous material (B) is then added to the solution, which is stirred preferably under a temperature of 5 to 60° C. for 1 to 24 hours, to prepare a mixture. The organic solvent is then removed from the mixture obtained by distillation and the like, and the remaining solids are dried under reduced pressure to obtain the carbon dioxide absorbent.

As the organic solvent, from a viewpoint of solubility of the amine compound (A) and from a viewpoint of easy removal from the carbon dioxide absorbent, preferable is dichloromethane or a monovalent alcohol having 4 or less of carbon atoms, and more preferable is at least one selected from the group consisting of dichloromethane, methanol, ethanol, and isopropyl alcohol.

### [Method for Capturing Carbon Dioxide]

A method for capturing carbon dioxide (hereinafter simply referred to as “the method of the present invention”, too) of the present invention is characterized by comprising using the above carbon dioxide absorbent.

The method of the present invention can elevate the amount of carbon dioxide absorbed from a gas containing carbon dioxide and can be suitably used, particularly, in technology of directly capturing carbon dioxide (DAC) from the air.

Carbon dioxide can be captured with lower energy, and the repeated usability of the carbon dioxide absorbent can be improved even in the case of using a steam stripping method in desorbing carbon dioxide from the absorbent with carbon dioxide absorbed.

It is preferable that the method for capturing carbon dioxide according to the present invention comprises a step of bringing the gas containing carbon dioxide into contact with the carbon dioxide absorbent, to cause the carbon dioxide absorbent to absorb carbon dioxide (absorption step).

### <Absorption Step>

The absorption step is a step of bringing the gas containing carbon dioxide into contact with the carbon dioxide absorbent, to cause the carbon dioxide absorbent to absorb the carbon dioxide. A contact method of the carbon dioxide absorbent and the gas can be selected as suitable one depending on morphology of the carbon dioxide absorbent and is not limited. For example, by passing the gas containing carbon dioxide through the carbon dioxide absorbent, spraying the carbon dioxide absorbent in the gas containing carbon dioxide, or locating the carbon dioxide absorbent in the gas containing carbon dioxide, the gas containing carbon dioxide can be brought into contact with the carbon dioxide absorbent.

The gas containing carbon dioxide is not limited, but includes, for example, air, thermal power plant exhaust gas, steel plant exhaust gas, cement plant exhaust gas, chemical plant exhaust gas, bio-fermentation gas, and natural gas. There is a need for capturing carbon dioxide from these gases with particularly energy savings, and the present invention is particularly effective. A concentration of carbon dioxide in the gas, pressure of the gas, and a temperature of the gas are not limited, and the method of the present invention can be applied in gases with a wide range of conditions.

In addition, the gas containing carbon dioxide may also contain acidic gas and the like other than carbon dioxide. The acidic gas includes CO, NOx, and SOx in the exhaust gas, formaldehyde come up from methanol-fueled power generation, and other hydrogen chloride, hydrogen sulfide. When the above gas containing carbon dioxide contains acidic gases and the like other than carbon dioxide, it is preferable that known steps for removing other acidic gases are combined. Specifically, included is an aspect that the method for capturing carbon dioxide of the present invention is applied to the gas containing acidic gases and the like other than carbon dioxide, or an aspect that the method for capturing carbon dioxide of the present invention is applied after removing other acidic gases from the gas containing acidic gases and the like other than carbon dioxide by a known means.

A relative humidity (RH) of the gas containing carbon dioxide is preferably 30% RH or more, more preferably 35% RH or more, and even more preferably 40% RH or more, and preferably 70% RH or less, more preferably 65% RH or less, and even more preferably 60% RH or less, from the viewpoint of improving carbon dioxide separating and capturing efficiency. In the present specification, a gas of less than 30% RH is defined as a gas in a dry state.

In the absorption step, a temperature when the gas containing carbon dioxide is brought into contact with the carbon dioxide absorbent is not limited, but is preferably 0° C. or more and less than 60° C., more preferably 20° C. or more and less than 60° C., from the viewpoint of improving the amount of carbon dioxide absorbed.

More preferably, the method of the present invention comprises the absorption step of bringing the gas containing carbon dioxide into contact with the carbon dioxide absorbent, to cause the carbon dioxide absorbent to absorb the carbon dioxide, and a desorption step of desorbing carbon dioxide from the carbon dioxide absorbent with carbon dioxide absorbed in the absorption step, wherein the desorption step comprises at least one step selected from the group consisting of the following (I) to (III). By this method, carbon dioxide can be easily separated and captured from the gas containing carbon dioxide.
(I) a step of subjecting the carbon dioxide absorbent with carbon dioxide absorbed to a reduced pressure condition;
(II) a step of bringing an inert gas not containing carbon dioxide into contact with the carbon dioxide absorbent with carbon dioxide absorbed; and
(III) a step of heating the carbon dioxide absorbent with carbon dioxide absorbed.

### <Desorption Step>

A desorption step is a step of desorbing carbon dioxide from the carbon dioxide absorbent with carbon dioxide absorbed in the absorption step. The method for desorbing carbon dioxide from the carbon dioxide absorbent with carbon dioxide absorbed preferably includes a method comprising at least one step selected from the group consisting of the above (I) to (III). Two or more of the steps (I) to (III) may be combined.

The desorption step preferably comprises the step (III) from the viewpoint of the effectiveness of use of the carbon dioxide absorbent of the present invention. The carbon dioxide absorbent of the present invention comprises the solid amine compound (A) and can therefore maintain high levels of a mass retention rate of a carbon dioxide absorbent and a retention rate of an amount of carbon dioxide absorbed even when heating is performed in the desorption step.

In the (I) step of subjecting the carbon dioxide absorbent with carbon dioxide absorbed to the reduced pressure condition (hereinafter referred to as “Step (I)”, too), the reduced pressure condition is preferably 10 kPa or less, more preferably 5 kPa or less, even more preferably 1 kPa or less, from a viewpoint of improving carbon dioxide separating and capturing efficiency.

In Step (I), a temperature when the carbon dioxide absorbent is subjected to the reduced pressure condition is not limited, but from the viewpoint of improving carbon dioxide separating and capturing efficiency, it is preferably 0° C. or more, and more preferably 10° C. or more. In addition, from the viewpoint of inhibiting the thermal deterioration of the amine compound (A) in the carbon dioxide absorbent, it is preferably less than 50° C., and more preferably 45° C. or less.

In the (II) step of bringing the inert gas not containing carbon dioxide into contact with the carbon dioxide absorbent with carbon dioxide absorbed (hereinafter referred to as “Step (II)”, too), reducing a partial pressure of carbon dioxide allows to promote the desorption of carbon dioxide. The inert gas not containing carbon dioxide includes nitrogen, helium, argon, and one or more of these gases can be used. Above all, from the viewpoint of improving the carbon dioxide separating and capturing efficiency, the inert gas not containing carbon dioxide is preferably at least one selected from the group consisting of the nitrogen and the argon.

In Step (II), the method for bringing the inert gas not containing carbon dioxide into contact with the carbon dioxide absorbent includes the same method as the contact method described in the absorption step.

In Step (II), a temperature when the inert gas not containing carbon dioxide is brought into contact with the carbon dioxide absorbent is not limited, and heating as specified in the step (III) may be simultaneously performed in Step (II), or may be at a temperature of room temperature or less. From the viewpoint of inhibiting the thermal deterioration of the amine compound (A) in the carbon dioxide absorbent, the temperature is preferably less than 50° C., and more preferably 45° C. or less. In addition, from the viewpoint of improving the carbon dioxide separating and capturing efficiency, the temperature is preferably 0° C. or more, and more preferably 10° C. or more.

A heating temperature in the (III) step of heating the carbon dioxide absorbent with carbon dioxide absorbed (hereinafter referred to as “Step (III)”, too) can be appropriately selected depending on the melting point and the maximum carbon dioxide release temperature of the carbon dioxide absorbent, but is preferably 50° C. or more, more preferably 55° C. or more, and even more preferably 60° C. or more, still more preferably 70° C. or more, and yet more preferably 80° C. or more, from the viewpoint of improving the carbon dioxide separating and capturing efficiency. The heating temperature is preferably 250° C. or less, more preferably 230° C. or less, even more preferably 220° C. or less, still more preferably 200° C. or less, and still more preferably 180° C. or less, from the viewpoint of improving the repeated usability of the carbon dioxide absorbent.

The heating in Step (III) can be performed by a known method using apparatus equipped with a heating means. A heating method includes, for example, heating with steam or a heating medium, hot air heating, electromagnetic wave heating, ultrasonic heating, and induction heating.

In the method of the present invention, the carbon dioxide absorbent comprising the amine compound (A) that is a solid and is low soluble in water is used. It is therefore preferable that a steam stripping method is used in Step (III). The steam stripping method can be performed by heating the carbon dioxide absorbent with carbon dioxide absorbed under a steam atmosphere. A heating temperature in the steam stripping method is preferably 80° C. or more, and more preferably 90° C. or more, and from the viewpoint of improving the repeated usability of the carbon dioxide absorbent, preferably 250° C. or less, more preferably 230° C. or less, even more preferably 220° C. or less, still more preferably 200° C. or less, and yet more preferably 180° C. or less.

The carbon dioxide absorbent and the carbon dioxide which are separated in the desorption step can be captured separately and reused.

### [Carbon Dioxide Separation and Capture Apparatus]

The carbon dioxide separation and capture apparatus of the present invention (hereinafter referred to as “the apparatus of the present invention”, too) comprises an absorption apparatus comprising a mechanism for bringing the gas containing carbon dioxide into contact with the carbon dioxide absorbent, to cause the carbon dioxide absorbent to absorb the carbon dioxide, and a desorption apparatus comprising a mechanism for desorbing carbon dioxide from the carbon dioxide absorbent with carbon dioxide absorbed.

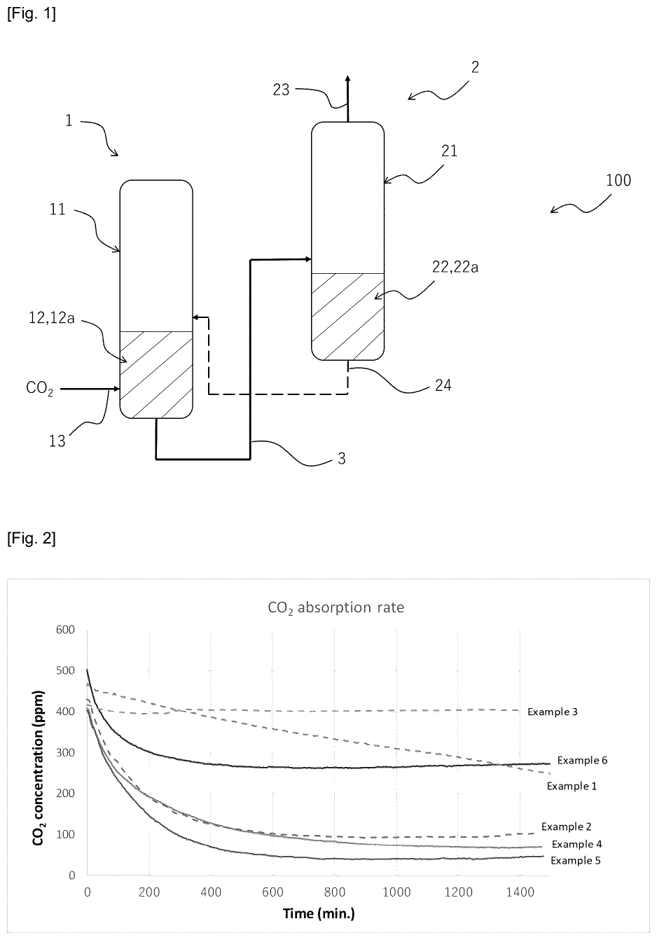

The apparatus of the present invention will be described with reference to FIG. 1. FIG. 1 is a schematic diagram showing an embodiment of a carbon dioxide separation and capture apparatus 100 of the present invention, in which 1 denotes the absorption apparatus and 2 denotes the desorption apparatus in FIG. 1.

### <Absorption Apparatus>

The absorption apparatus 1 in the carbon dioxide separation and capture apparatus 100 is equipped with the carbon dioxide absorbent and comprises a mechanism for bringing the gas containing carbon dioxide into contact with the carbon dioxide absorbent, to cause the carbon dioxide absorbent to absorb the carbon dioxide.

The absorption apparatus 1 is not limited, as long as it has configuration such that the gas containing carbon dioxide are brought into contact with the carbon dioxide absorbent, depending on the morphology of the carbon dioxide absorbent. As shown in FIG. 1, for example, the absorption apparatus 1 can be equipped with an absorbent holding part 12 that holds a carbon dioxide absorbent 12a inside a reaction column 11 and can be further equipped with a gas supplying part 13 that supplies the gas containing carbon dioxide to the absorbent holding part 12. In addition, the absorption apparatus 1 can be also equipped with an absorbent discharging part (not shown) that discharges the carbon dioxide absorbent 12a held in the absorbent holding part 12 and an absorbent supplying part (not shown) that supplies a new carbon dioxide absorbent to the absorbent holding part 12, from a viewpoint of discharging the carbon dioxide absorbent with carbon dioxide absorbed from the absorbent holding part 12 and supplying a new carbon dioxide absorbent.

The absorption apparatus 1 may further comprise a heating and cooling mechanism to adjust the temperature when the gas containing carbon dioxide is brought into contact with the carbon dioxide absorbent. It may also comprise a carbon dioxide concentration measuring mechanism so that a carbon dioxide concentration in the gas can be measured. Furthermore, the absorption apparatus 1 may comprise a pressurizing and depressurizing mechanism to adjust pressure when the gas containing carbon dioxide is brought into contact with the carbon dioxide absorbent.

The carbon dioxide separation and capture apparatus 100 may have a connecting part 3 for supplying the carbon dioxide absorbent with carbon dioxide absorbed in the absorption apparatus 1 to the desorption apparatus 2.

A means of supplying the carbon dioxide absorbent with carbon dioxide absorbed from the absorption apparatus 1 to the desorption apparatus 2 is not limited, and the absorption apparatus 1 operated for a certain period of time may be stopped once, and the carbon dioxide absorbent in the reaction column 11 equipped in the absorption apparatus 1 may be supplied to the desorption apparatus 2 together. Alternatively, from the absorbent holding part 12 of the absorption apparatus 1, the carbon dioxide absorbent may be supplied continuously or interruptedly to the desorption apparatus 2 by utilizing the connecting part 3.

### <Desorption Apparatus>

The desorption apparatus 2 in the carbon dioxide separation and capture apparatus 100 comprises a mechanism for desorbing carbon dioxide from the carbon dioxide absorbent with carbon dioxide absorbed in the absorption apparatus 1. The desorption apparatus 2 is not limited as long as it comprises a mechanism for desorbing carbon dioxide from the carbon dioxide absorbent with carbon dioxide absorbed in the absorption apparatus 1, but it is preferable that it comprises a mechanism to perform at least one step of the above (I) to (III). Examples of the mechanism include a depressurizing mechanism, an inert gas supplying mechanism, and a heating mechanism.

As shown in FIG. 1, for example, the desorption apparatus 2 can be equipped with an absorbent holding part 22 that holds a carbon dioxide absorbent 22a with carbon dioxide absorbed inside a reaction column 21, and further equipped with a gas discharging part 23 that discharges carbon dioxide desorbed from the carbon dioxide absorbent. The desorption apparatus 2 also comprises at least one mechanism (not shown) selected from a depressurizing mechanism for bringing the inside of the reaction column 21 under a reduced pressure condition, an inert gas supplying mechanism for supplying the inert gas to the absorbent holding part 22, and a heating mechanism for heating the absorbent holding part 22.

In addition to the depressurizing mechanism, the inert gas supplying mechanism, and the heating mechanism described above, the desorption apparatus 2 may comprise the carbon dioxide concentration measuring mechanism or the like, in the same manner as the absorption apparatus 1.

The carbon dioxide absorbent after carbon dioxide is desorbed in the desorption apparatus 2 can also be reused by supplying it back into the absorption apparatus 1 again from an absorbent discharging part 24 for supplying the carbon dioxide absorbent after carbon dioxide is desorbed to the absorption apparatus 1.

The carbon dioxide separation and capture apparatus 100 may further comprise a capturing apparatus for capturing carbon dioxide desorbed. Still, here, the carbon dioxide captured may be used for agricultural applications such as a petroleum promoted capturing method and a plant factory; industrial gas applications such as beverages and welding; chemical synthesis applications; and carbon dioxide capture and storage (CCS) applications. Prior to use in these applications, the carbon dioxide captured may also be concentrated.

### EXAMPLES

Hereinafter, the present invention will be described with reference to examples, but the present invention is not limited to the scope of the examples. In the present examples, various measurements and evaluations were performed by the following methods.

### (Specific Surface Area and Pore Volume of Porous Material)

The specific surface area and the pore volume of the porous material were measured by the specific surface area/pore size distribution measurement analyzer (“ASAP2020” produced by Shimadzu Corporation).

### (Volume Median Particle Size (D₅₀) of Porous Material)

Using a laser diffraction/scattering particle size distribution analyzer (“LMS-200e” produced by Malvern Panalytical Ltd.), particle distribution of the porous material was measured.

Then, the particle diameter corresponding to 50% of a cumulative volume frequency calculated from the smaller particle diameter of the particle distribution was taken as the volume median particle size (D₅₀) of the porous material.

### (Carbon Dioxide (CO₂) Absorption Property of Carbon Dioxide Absorbent)

A carbon dioxide concentration meter and a petri dish were placed inside an openable desiccator (Internal Dimension: 370 mm×260 mm×272 mm) under an air environment of 23° C. and 50% RH. The carbon dioxide absorbent was then added in an amount of 10 mmol in terms of the number of amino groups in the amine compound (5 mmol as the amine compound in the present Examples) contained in the carbon dioxide absorbent to the petri dish in the desiccator, a door was immediately closed, and the carbon dioxide absorbent was stood in the desiccator.

After a lapse of a given time, a carbon dioxide concentration (ppm) in the desiccator was measured with the CO₂ concentration meter (“CO2/Temperature/Humidity Data Logger TR-76Ui” produced by T&D Corporation), and the carbon dioxide absorption property of the carbon dioxide absorbent was evaluated from the concentration. FIG. 2 shows a graph in which the carbon dioxide concentration (ppm) in the desiccator was plotted on the ordinate against an elapsed time (min) on the abscissa. A larger amount of decrease in carbon dioxide concentration means a higher carbon dioxide absorption property of the carbon dioxide absorbent.

### (Maximum Carbon Dioxide (CO₂) Release Temperature of Carbon Dioxide Absorbent)

A carbon dioxide concentration meter and a petri dish were placed inside an openable desiccator (Internal Dimension: 370 mm×260 mm×272 mm) under an air environment of 23° C. and 50% RH. The carbon dioxide absorbent was then added in an amount of 10 mmol in terms of the number of amino groups in the amine compound (5 mmol as the amine compound in the present Examples) contained in the carbon dioxide absorbent to the petri dish in the desiccator, a door was immediately closed, and the carbon dioxide absorbent was stood in the desiccator for 24 hours. An initial concentration of carbon dioxide was adjusted to about 400 ppm.

Then, the carbon dioxide absorbent was removed from the desiccator to obtain the carbon dioxide absorbent with carbon dioxide absorbed. The DSC measurement was performed on the carbon dioxide absorbent with carbon dioxide absorbed as follows, to measure the maximum carbon dioxide release temperature of the carbon dioxide absorbent.

First, the differential scanning calorimetry was performed on the carbon dioxide absorbent using the differential thermogravimetric analyzer (“DTG-60” produced by Shimadzu Corporation) under the conditions of 23 to 250° C. of a measurement temperature range, 10° C./minute of the heating rate, and the nitrogen atmosphere. From the DSC curve obtained thereby, the temperature at which the endothermic amount associated with the desorption of carbon dioxide reached the maximum was used as the maximum carbon dioxide release temperature of the carbon dioxide absorbent.

### (Melting Point of Carbon Dioxide Absorbent)

DSC measurement was carried out using a simultaneous thermogravimetry/differential thermal analyzer (“STA200RV” produced by Hitachi, Ltd.). The measurement conditions were set to a nitrogen flow rate of 50 mL/minute, a heating rate of 10° C./minute, and a measurement temperature range of 40 to 350° C., and about 16 mg of the carbon dioxide absorbent was weighed for measurement. The maximum endothermic peak temperature in the obtained DSC chart was used as the melting point.

(Moisture Content of Carbon Dioxide Absorbent after Carbon Dioxide Absorption)

Measurement was performed using an autosampler-type moisture meter (“CA-310” produced by Nitto Seiko Analytech Co., Ltd.). The amount of a sample was 0.1 g, the measurement conditions were set to a vaporization temperature of 105° C. and a heating time of 20 minutes, and a value of (the amount of decrease in sample weight)/(the sample weight before measurement)×100(%) was used as the moisture content.

### (Solubility of Amine Compound in Water)

An aqueous solution was adjusted to 0.1% by mass of the concentration of the amine compound, and whether or not the amine compound was dissolved at 25° C. was visually observed. The solubility was determined as “present” when no undissolved residue of the amine compound was seen, and as “absent” when the undissolved residue was seen. The results are shown in Examples 1 to 3 of Table 2.

### (Cycle Test)

A cycle test of CO₂ absorption-desorption of the carbon dioxide absorbent was conducted by temperature programs shown in Table 1 using a TG-DTA apparatus (“STA200RV” produced by Hitachi, Ltd.). About 16 mg of the carbon dioxide absorbent was placed in a deep aluminum pan, which was then loaded in the TG-DTA apparatus and subjected to the temperature history of temperature programs Nos. 1 to 11 described in Table 1 to conduct the cycle test of CO₂ absorption-desorption. A measurement atmosphere during the run of each program is shown in the column “Gas” in Table 1, and the flow rate of the gas is shown in the column “Gas Flow Rate”. Moisture-Absorbed Air in Table 1 means air (30 to 70% RH) subjected to moisture control by passing dry air after a drying process through water.

X (° C.) in Table 1 corresponds to a regeneration temperature (° C.) in the cycle test described later.

TABLE 1
Table 1

| | Start | End | Heating | Retention | | Gas Flow | |
|---|---|---|---|---|---|---|---|
| Program | Temperature | Temperature | Rate | Time | Gas | Rate | |
| No. | (° C.) | (° C.) | (° C./min.) | (min.) | — | (mL/min.) | Remarks |
| 1 | 40 | X | 5 | 30 | N₂ | 50 | Adsorption Gas |
| | | | | | | | and Moisture |
| 2 | X | 40 | −5 | 2 | N₂ | 50 | Removal |
| 3 | 40 | 40 | — | 360 | Moisture- | 200 | First Run of CO₂ |
| | | | | | Absorbed | | Absorption |
| | | | | | Air | | |
| 4 | 40 | X | 5 | 30 | N₂ | 50 | First Run of CO₂ |
| 5 | X | 40 | −5 | 2 | N₂ | 50 | Desorption |
| 6 | 40 | 40 | — | 360 | Moisture- | 200 | Second Run of |
| | | | | | Absorbed | | CO₂ Absorption |
| | | | | | Air | | |
| 7 | 40 | X | 5 | 30 | N₂ | 50 | Second Run of |
| 8 | X | 40 | −5 | 2 | N₂ | 50 | CO₂ Desorption |
| 9 | 40 | 40 | — | 360 | Moisture- | 200 | Third Run of |
| | | | | | Absorbed | | CO₂ Absorption |
| | | | | | Air | | |
| 10 | 40 | X | 5 | 30 | N₂ | 50 | Third Run of |
| 11 | X | 40 | −5 | 2 | N₂ | 50 | CO₂ Desorption |

[Amount of CO₂ Absorbed (mg/g) at Second Cycle]

The amount (mg) of increase in weight of the absorbent at the completion of program No. 6 in Table 1 was divided by the weight (g) of the absorbent at the start of program No. 6, and the obtained value was used as the amount of CO₂ absorbed (mg/g) at the second cycle. A larger value means a better carbon dioxide absorption property.

### [Regeneration Rate (%) at Second Cycle of CO₂ Absorption-Desorption]

The weight (g) of the absorbent at the start of program No. 6 in Table 1 was divided by the weight (g) of the absorbent at the completion of program No. 8, and the obtained value was multiplied by 100 and used as the regeneration rate (%) at the second cycle of CO₂ absorption-desorption. A higher regeneration rate means better repeated usability.

### [Mass Retention Rate (Second Cycle/Third Cycle) (%)]

The weight (g) of the absorbent at the completion of program No. 11 in Table 1 was divided by the weight (g) of the absorbent at the completion of program No. 8, and the obtained value was multiplied by 100 and used as the mass retention rate (second cycle/third cycle) (%). A higher mass retention rate means better repeated usability.

### [Retention Rate of Amount of CO₂ Absorbed (Second Cycle/Third Cycle) (%)]

The amount (mg) increase in weight of the absorbent at the completion of program No. 6 in Table 1 was divided by the weight (g) of the absorbent at the start of program No. 6, and the obtained value was used as the amount of CO₂ absorbed, Y, (mg/g) at the second cycle. The amount (mg) increase in weight of the absorbent at the completion of program No. 9 in Table 1 was divided by the weight (g) of the absorbent at the start of program No. 9, and the obtained value was used as the amount of CO₂ absorbed, Z, (mg/g) at the third cycle. The value of Z/Y×100 was used as the retention rate of the amount of CO₂ absorbed (second cycle/third cycle) (%). A higher retention rate means better repeated usability.

In Examples and Comparative Examples, as the amine compounds and the porous materials, the following were used.

### (Amine Compound)

HQ-BA: the compound represented by the following formula (1-1) (C₂₀H₂₀N₂O₂, Mw: 320.4). This compound was produced by a method described in Production Example 1 described later.

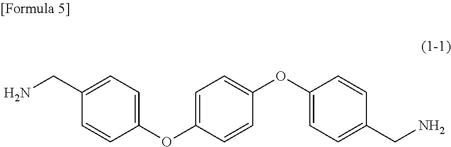 [SMILES: NCc1ccc(Oc2ccc(Oc3ccc(CN)cc3)cc2)cc1]

DAMB: the compound represented by the following formula (1-2) (C₁₄H₁₆N₂, Mw: 212.3). This compound was produced by a method described in Production Example 2 described later.

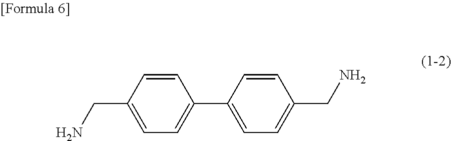 [SMILES: NCc1ccc(-c2ccc(CN)cc2)cc1]

TP-DMA: the compound represented by the following formula (1-3) (C₂₀H₂₀N₂, Mw: 288.4). This compound was synthesized in accordance with the method described in J. Am. Chem. Soc., No. 136, Vol. 22, p. 7899-7906 (2014).

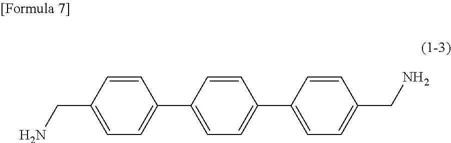 [SMILES: NCc1ccc(-c2ccc(-c3ccc(CN)cc3)cc2)cc1]

### Production Example 1 (Production of HQ-BA)

In accordance with the method described in Polymer Degradation and Stability, Vol. 95, p. 2445-2452 (2010), hydroquinone and 4-chlorobenzonitrile (4CBN) were reacted to synthesize a dinitrile compound (HQ-BEN) represented by the structural formula given below. Then, HQ-BEN was hydrogenated by the following method to produce HQ-BA.

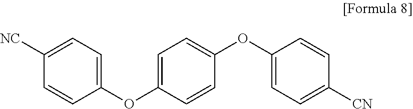 [SMILES: N#Cc1ccc(Oc2ccc(Oc3ccc(C#N)cc3)cc2)cc1]

2.99 g of a particulate Raney cobalt catalyst was weighed in water, then transferred to a 200 cc autoclave, and replaced with isopropyl alcohol three times. After removal of the isopropyl alcohol used in replacement, 15 g of a solvent metaxylene and 6 g of HQ-BEN were added. The autoclave was airproofed, and the inside of the autoclave was then purged with nitrogen (1 MPaG) and hydrogen (5 MPaG) three times each.

The autoclave was further charged with 15 g of liquid ammonia, and hydrogen was enclosed therein at 5 MPaG (including the liquid ammonia's own pressure). Heating was started with stirring at about 750 rpm, and reaction was performed at 80° C. The reaction time was 3 hours from the start of heating. After the completion of the reaction, the mixture was cooled with ice water, and the internal pressure of the system was reduced by purging with hydrogen and ammonia, followed by the opening of the autoclave. The reaction liquid was washed with methanol to remove the catalyst, and the solvent was then distilled off with an evaporator to obtain the target product HQ-BA.

### Production Example 2 (Production of DAMB)

4,4′-Dicyanobiphenyl (DCB, reagent) was hydrogenated by the following method to produce 4,4-di(aminomethyl) biphenyl (DAMB).

3.0 g of a particulate Raney cobalt catalyst was weighed in water, then transferred to a 100 mL autoclave, and reduced at normal pressure under hydrogen flow at 270° C. for 2 hours. Next, the catalyst was transferred to a 200 mL autoclave purged with argon, and a punching metal for fixing the catalyst was installed therein. 10 g of a solvent metaxylene and 1.5 g of a substrate DCB were added thereto, the autoclave was airproofed, and the inside of the autoclave was then purged with nitrogen (1 MPaG) and hydrogen (10 MPaG) three times each.

The autoclave was further charged with 10 g of liquid ammonia, and hydrogen was enclosed therein at 10 MPaG (including the liquid ammonia's own pressure). Heating was started with stirring at about 750 rpm, and reaction was performed for 1.5 hours after the internal temperature reached 80° C. After the completion of the reaction, the autoclave was cooled with ice water, and the internal pressure of the system was reduced by purging with hydrogen and ammonia, followed by the opening of the autoclave. The reaction liquid was washed with methanol to remove the catalyst, and the solvent was then distilled off with an evaporator to obtain the target product DAMB.

MXDA: metaxylylenediamine (produced by Mitsubishi Gas Chemical Co., Inc.); diamine liquid at 25° C., represented by the following formula.

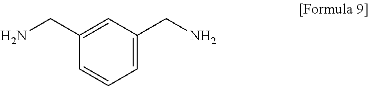

Spirobisamine: 2,2′-(2,4,8,10-tetraoxaspiro[5.5]undecane-3,9-diyl)bis(2-methylpropan-1-amine) produced by the method described in Example 1 of WO 2022/255037, represented by the following formula.

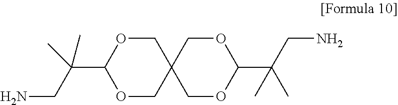 [SMILES: CC(C)(CN)C1OCC2(CO1)COC(C(C)(C)CN)OC2]

### (Porous Material)

### Mesoporous Silica SBA15 (Produced by Merck KGaA)

The specific surface area by the BET method: 800 m²/g, the volume median particle size (D₅₀): 100 μm, the pore volume: 0.8 cm³/g

### Pellet Silica (Pellets Obtained by Molding a Starting Material Silica Given Below)

The specific surface area by the BET method: 253 m²/g, the average pellet size: 1.90 mm, the average pellet length: 10.86 mm

Starting material silica (“CARiACT G-10” produced by Fuji Silysia Chemical Ltd., the specific surface area: 300 m²/g, the particle size: 10 μm, the pore volume: 1.3 mL/g, the average pore size: 10 nm)

### Examples 1 to 3 (Evaluation of Carbon Dioxide Absorbent)

FIG. 2 shows results of evaluating the carbon dioxide absorption property by the above method using the amine compounds shown in Table 2 as carbon dioxide absorbents. Table 2 shows results of evaluating the maximum carbon dioxide release temperature, the melting point, and the solubility in water by the above methods.

### Examples 4 to 6 (Preparation and Evaluation of Carbon Dioxide Absorbent)

### (Preparation of Carbon Dioxide Absorbent)

A glass container purged with nitrogen was charged with 1.5 g of the amine compound (A) described in Table 2, and a large excess of methanol (dichloromethane in Examples 5 and 6), and stirred with a stirrer at room temperature (23° C.) to dissolve the amine compound (A). Then, 1.5 g of the porous material was added thereto, and the container was purged with nitrogen again and stirred for 3 hours for homogenization. From the obtained mixture, methanol or dichloromethane was distilled off under reduced pressure of 50 hPa at 40° C. and then dried in vacuum at 80° C. for 3 hours to obtain a carbon dioxide absorbent having the amine compound (A) supported on the porous material (B).

### (Evaluation of Carbon Dioxide Absorbent)

FIG. 2 shows results of evaluating the carbon dioxide (CO₂) absorption property by the above method using the obtained carbon dioxide absorbents. Table 2 shows results of measuring the maximum carbon dioxide (CO₂) release temperature, the melting point, and the moisture content after carbon dioxide absorption by the above methods.

### Comparative Example 1

MXDA (metaxylylenediamine) was used as the carbon dioxide absorbent instead of the amine compound (A) in Example 1, and the maximum carbon dioxide release temperature, the melting point, and the solubility in water were evaluated by the above methods. As a result, the maximum carbon dioxide release temperature was 135° C., the melting point was 14° C., and the solubility in water was “present”. Since the carbon dioxide absorbent of Comparative Example 1 is a liquid at 25° C. and is dissolved in water, it is considered that MXDA is easily eluted into steam by the application of the steam stripping method during regeneration of the absorbent and good repeated usability cannot be obtained.

### Comparative Example 2

The spirobisamine was used as the carbon dioxide absorbent instead of the amine compound (A) in Example 1, and the carbon dioxide (CO₂) absorption property was evaluated by the above method. However, the spirobisamine exhibited no carbon dioxide absorption property.

### Comparative Example 3

The preparation of a carbon dioxide absorbent having the spirobisamine supported on the porous material (B) was attempted by the same method as in Example 4 except that the spirobisamine was used instead of the amine compound (A) in Example 4. However, the spirobisamine rarely has solvent solubility and therefore, was not able to be supported on the porous material (B).

TABLE 2
Table 2

| | | Example | | | | | |
|---|---|---|---|---|---|---|---|
| (% by Mass) | | 1 | 2 | 3 | 4 | 5 | 6 |
| Amine | HQ-BA | 100 | | | 50 | | |
| Compound (A) | DAMB | | 100 | | | 50 | |
| | TP-DMA | | | 100 | | | 50 |
| Porous Material | Mesoporous | | | | 50 | 50 | 50 |
| (B) | Silica | | | | | | |

| | | | | | | |
|---|---|---|---|---|---|---|
| Total | 100 | 100 | 100 | 100 | 100 | 100 |
| Maximum CO₂ Release Temperature (° C.) | 120 | 142 | 265 | 110 | 150 | 110 |
| Melting Point (° C.) | 131 | 148 | 282 | 132 | 150 | 266 |
| Moisture Content after Carbon | — | — | — | 1.9 | 1.9 | 2.4 |
| Dioxide Absorption (%) | | | | | | |
| Solubility in Water | Absent | Absent | Absent | — | — | — |

As seen from FIG. 2, the carbon dioxide absorbents of the present Examples exhibited the carbon dioxide absorption property even in the system containing carbon dioxide at low concentrations (0.04 to 0.05% by mass). It is evident by the comparison of Examples 1 to 3 with Examples 4 to 6 that the amine compounds (A) used in Examples 1 to 3 are supported on the porous material (B) and thereby further improve the carbon dioxide absorption property. This is presumably because the surface area of a portion capable of coming into contact with carbon dioxide is widened in the amine compound (A) having the carbon dioxide absorption property.

As seen from Table 2, it is evident by the comparison of Example 1 with Example 4 and the comparison of Example 3 with Example 6 that the amine compounds (A) used in Examples 1 and 3 are supported on the porous material (B) and thereby largely decrease the maximum CO₂ release temperature and also increase the difference between the maximum CO₂ release temperature and the melting point. This facilitates desorbing carbon dioxide at a temperature lower than the melting point of the carbon dioxide absorbent and can circumvent reduction in carbon dioxide absorption property caused by the melting of the carbon dioxide absorbent.

All the carbon dioxide absorbents of the present Examples have low solubility in water or a low water absorption property and are therefore expected to have good repeated usability because the amine compound (A) is difficult to elute into steam even by the application of the steam stripping method during regeneration of the absorbent.

The carbon dioxide absorbents of Examples 4 to 6 containing the amine compounds (A) of Examples 1 to 3 were subjected to the cycle test by the above method. The results are shown in Table 3.

TABLE 3
Table 3

| Carbon Dioxide Absorbent | — | Example 4 | | | Example 5 | | Example 6 | |
|---|---|---|---|---|---|---|---|---|
| Regeneration Temperature X | ° C. | 90 | 120 | 150 | 120 | 170 | 90 | 130 |
| Maximum CO₂ Release | ° C. | 110 | 110 | 110 | 150 | 150 | 110 | 110 |
| Temperature | | | | | | | | |
| Amount of CO₂ Absorbed at | mg/g | 16.9 | 16.0 | 16.9 | 73.9 | 88.3 | 28.1 | 27.0 |
| Second Cycle of CO₂ | | | | | | | | |
| Absorption-Desorption | | | | | | | | |
| Regeneration Rate at Second | % | >99 | >99 | >99 | >99 | >99 | >99 | >99 |
| Cycle of CO₂ Absorption- | | | | | | | | |
| Desorption | | | | | | | | |
| Mass Retention Rate (Second | % | >99 | >99 | >99 | >99 | 95 | >99 | >99 |
| Cycle/Third Cycle) | | | | | | | | |
| Retention Rate of Amount of | % | 99.3 | 98.0 | 96.8 | 93.0 | 97.1 | 95.1 | 94.7 |
| CO₂ Absorbed (Second | | | | | | | | |
| Cycle/Third Cycle) | | | | | | | | |

As seen from Table 3, it is evident that the carbon dioxide absorbents of the present Examples have both a high mass retention rate of the carbon dioxide absorbent and a high retention rate of the amount of carbon dioxide absorbed even after the cycle of carbon dioxide absorption and carbon dioxide desorption by heating is repeated.

### Examples 7 and 8 (Preparation and Evaluation of Carbon Dioxide Absorbent)

### (Preparation of Carbon Dioxide Absorbent)

A glass container purged with nitrogen was charged with 8 g of HQ-BA as the amine compound (A) and 500 g of methanol, and stirred with a stirrer at room temperature (23° C.) to dissolve the amine compound (A). Then, 12 g of pellet silica as the porous material (B) was added thereto, and the container was purged with nitrogen again and stirred for 3 hours for homogenization. From the obtained mixture, methanol was distilled off under reduced pressure of 50 hPa at 40° C. and then dried in vacuum at 80° C. for 3 hours to obtain a carbon dioxide absorbent A having the amine compound (A) supported on the porous material (B) and having an amine compound (A) content of 40% by mass.

### (Evaluation of Carbon Dioxide Absorbent)

The cycle of performing a carbon dioxide absorption operation described later using the obtained carbon dioxide absorbent A and then performing a regeneration operation of the carbon dioxide absorbent A described later was repeated a total of three times to conduct the cycle test of CO₂ absorption-desorption of the carbon dioxide absorbent. The obtained results are shown in Table 4 about the amount of carbon dioxide absorbed.

### [Carbon Dioxide Absorption Operation]

In Example 7, first, dry simulant air (0% RH) having a CO₂ concentration of 400 ppm was prepared using an air tank gas and a CO₂/N₂ tank gas. This dry simulant air was circulated at 5 L/minute in a column (diameter: 30 mm, length: 10 cm) packed with 10 g of the carbon dioxide absorbent A, and a carbon dioxide concentration (ppm) was measured at each of the entry and the outlet for the dry simulant air in this column using a NDIR (non-dispersive infrared) CO₂ concentration meter (produced by Vaisara). The amount of carbon dioxide absorbed by the carbon dioxide absorbent A was calculated from an accumulated value of difference in measured carbon dioxide concentration between the entry and the outlet of the column within a predetermined time (300 minutes) when the dry simulant air was circulated.

In Example 8, first, an air tank gas was circulated in water to provide a high-moisture gas having a humidity of 100% RH, and a CO₂/N₂ tank gas and an air tank gas were mixed and adjusted such that a CO₂ concentration was a concentration of 800 ppm to provide a high-CO₂ gas. These gases were further mixed at a volume ratio of 1:1 to prepare moisture-controlled simulant air (50% RH) having a CO₂ concentration of 400 ppm. This moisture-controlled simulant air was circulated at 5 L/minute in a column (diameter: 30 mm, length: 10 cm) packed with 10 g of the carbon dioxide absorbent A, and a carbon dioxide concentration (ppm) was measured at each of the entry and the outlet for the moisture-controlled simulant air in this column using an NDIR (non-dispersive infrared) CO₂ concentration meter (produced by Vaisara). The amount of carbon dioxide absorbed by the carbon dioxide absorbent A was calculated from an accumulated value of difference in measured carbon dioxide concentration between the entry and the outlet of the column within a predetermined time (300 minutes) when the moisture-controlled simulant air was circulated.

### [Regeneration Operation of Carbon Dioxide Absorbent]

After the above absorption operation, the pressure of the whole column was reduced to 47.4 kPa, and steam of 80° C. was circulated in the column for 10 minutes so that carbon dioxide adsorbed to the carbon dioxide absorbent A was desorbed. While the internal temperature of the column was then held at 80° C., a dry nitrogen gas was circulated at 5 L/minute for 30 minutes to dry the carbon dioxide absorbent A.

TABLE 4
Table 4

| | Example 7 | | | Example 8 | | |
|---|---|---|---|---|---|---|
| Amine | HQ-BA | | | | | |
| Compound (A) | | | | | | |
| Porous | Pellet Silica | | | | | |
| Material (B) | | | | | | |
| Gas | Dry Simulant Air | | | Moisture-Controlled Simulant Air (50% RH) | | |
| Amount of | Cycle 1 | Cycle 2 | Cycle 3 | Cycle 1 | Cycle 2 | Cycle 3 |
| CO₂ Absorbed | 2.2 | 4.3 | 3.4 | 4.6 | 7.3 | 8.1 |

From Table 4, it is evident that the carbon dioxide absorbents of the present Examples have a higher amount of carbon dioxide absorbed when the gas containing carbon dioxide has a high humidity than when this gas is in a dry state.

### INDUSTRIAL APPLICABILITY

According to the present invention, a carbon dioxide absorbent excellent in carbon dioxide absorption property and repeated usability, a method for capturing carbon dioxide using the same, and a carbon dioxide separation and capture apparatus can be provided.

### REFERENCE SIGNS LIST

100 carbon dioxide separation and capture apparatus
1 absorption apparatus
2 desorption apparatus
3 connecting part
11, 21 reaction column
12, 22 absorbent holding part
12a carbon dioxide absorbent
13 gas supplying part
21 reaction column
22a carbon dioxide absorbent with carbon dioxide absorbed
23 gas discharging part
24 absorbent discharging part

1. A carbon dioxide absorbent comprising a non-azo solid amine compound (A) having two or more aromatic rings and having two or more primary amino groups.

2. The carbon dioxide absorbent according to claim 1, wherein the amine compound (A) is a non-polymer compound.

3. The carbon dioxide absorbent according to claim 1, wherein the amine compound (A) is an amine compound represented by the following general formula (1): 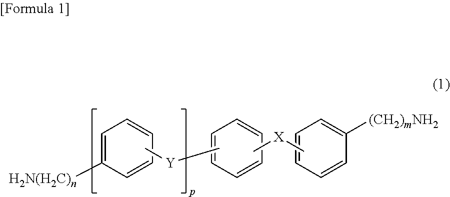
wherein in the formula (1), X and Y each independently represent a single bond or an oxygen atom; n and m each independently represent a number of 1 to 6; and p represents a number of 0 to 2.

4. The carbon dioxide absorbent according to claim 3, wherein the amine compound represented by the general formula (1) is a compound represented by any of the following formulas (1-1) to (1-3):

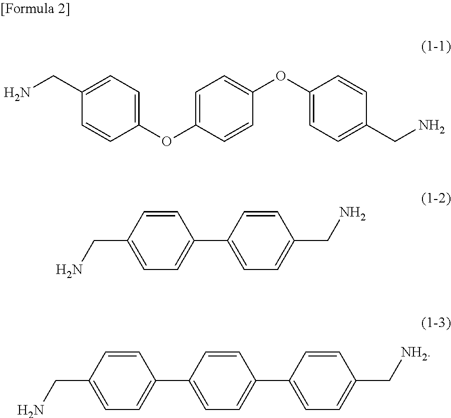

5. The carbon dioxide absorbent according to claim 1, wherein the carbon dioxide absorbent comprises the amine compound (A) and a porous material (B), and at least some of the amine compound (A) is supported on the porous material (B).

6. The carbon dioxide absorbent according to claim 5, wherein the porous material (B) comprises at least one selected from the group consisting of porous silica and porous alumina.

7. A method for capturing carbon dioxide, comprising using the carbon dioxide absorbent according to claim 1.

8. The method according to claim 7, wherein the method comprises:
absorption by bringing a gas containing carbon dioxide into contact with the carbon dioxide absorbent, to cause the carbon dioxide absorbent to absorb the carbon dioxide, and
desorption by desorbing carbon dioxide from the carbon dioxide absorbent with carbon dioxide absorbed in the absorption, and
wherein the desorption comprises at least one selected from the group consisting of the following (I) to (III):
(I) subjecting the carbon dioxide absorbent with carbon dioxide absorbed to a reduced pressure condition;
(II) bringing an inert gas not containing carbon dioxide into contact with the carbon dioxide absorbent with carbon dioxide absorbed; and
(III) heating the carbon dioxide absorbent with carbon dioxide absorbed.

9. A carbon dioxide separation and capture apparatus comprising: an absorption apparatus comprising a mechanism for bringing a gas containing carbon dioxide into contact with the carbon dioxide absorbent according to claim 1, to cause the carbon dioxide absorbent to absorb the carbon dioxide; and a desorption apparatus comprising a mechanism for desorbing carbon dioxide from the carbon dioxide absorbent with carbon dioxide absorbed.
